(19) United States
(12) Patent Application Publication
(10) Pub. No.: US 2026/0199584 A1
(43) Pub. Date: Jul. 16, 2026

(54) NEGATIVE PRESSURE WOUND THERAPY SYSTEM

(71) Applicant: Solventum Intellectual Properties Company, Maplewood, MN (US)

(72) Inventors: Benjamin A. PRATT, Poole (GB); Robert HOWARD, Westmeath (GB); Michael R. OLIVA, Saint Paul, MN (US); Larry Tab RANDOLPH, San Antonio, TX (US); Richard M. KAZALA, JR., San Antonio, TX (US); James SEDDON, Wimborne (GB)

(21) Appl. No.: 19/134,004

(22) PCT Filed: Dec. 5, 2023
(86) PCT No.: PCT/IB2023/062237

§ 371 Date: May 29, 2025

Related U.S. Application Data
(60) Provisional application No. 63/435,349, filed on Dec. 27, 2022.

(51) Int. Cl. A61M 1/00; A61M 39/24

(52) U.S. Cl. CPC A61M 1/966; A61M 39/24; A61M 2039/242; A61M 2205/3327; A61M 2205/3344; A61M 2205/8206

(57) ABSTRACT

A negative pressure wound therapy (NPWT) system for treatment of a wound site includes a therapy unit including a housing, a negative pressure source disposed within the housing, and a first printed circuit board disposed within the housing. The NPWT system includes a battery pack including a second printed circuit board. The NPWT system further includes a power connector assembly, a wound dressing coupled to the wound site and disposed in fluid communication with the therapy unit, and an ambient pressure sensor. The ambient pressure sensor is configured to generate a signal indicative of an ambient pressure. The first printed circuit board is communicably coupled to the ambient pressure sensor for receiving the signal from the ambient pressure sensor.

### CROSS-REFERENCE TO RELATED APPLICATIONS

This application is a U.S. National Stage Entry of PCT International Application No. PCT/IB2023/062237, filed on Dec. 5, 2023, which claims the benefit of priority to U.S. Provisional Application No. 63/435,349, filed on Dec. 27, 2022, which is incorporated herein by reference in its entirety.

### TECHNICAL FIELD

The present disclosure relates generally to a negative pressure wound therapy system for treatment of a wound site, and a method for providing negative pressure wound therapy to the wound site.

### BACKGROUND

Negative pressure wound therapy (NPWT) systems are embodied as sealed wound-care systems particularly indicated for wounds, such as, chronic persistent wounds and/or complicated wounds. Specifically, for promoting wound healing, a pressure that is reduced relative to the surroundings (commonly referred to as “negative pressure”) is applied to the wound. The negative pressure causes mechanical contraction of the wound and removal of exudates, such as, slough, necrotic tissue, and microbial load (e.g., bacteria and biofilms), from the wound, thus promoting formation of granulation tissues and accelerating wound healing.

Conventional NPWT systems include a therapy device that is in fluid communication with the wound. Further, the NPWT system includes an exudate cannister to collect exudates removed from the wound. The therapy device includes a negative pressure source, such as a pump, that applies the negative pressure to the wound. The therapy device also includes a pressure sensor that provides pressure readings so that the negative pressure can be applied to the wound. However, such a pressure sensor may only be useful to administer negative pressure therapy for a short duration. For long duration negative pressure therapy applications or when users are mobile, there may be changes in ambient pressures. In such cases, an additional pressure sensor may be required. Usage of multiple pressure sensors may increase cost and complexity of the therapy device. It may be desirable to design the therapy device and incorporate the pressure sensor in the therapy device in such a way that the therapy device is compact in size, easy to use, cost-effective, and provides a sustainable solution with minimum environmental impact.

### SUMMARY

Generally, the present disclosure relates to a negative pressure wound therapy system for treatment of a wound site, and a method for providing negative pressure wound therapy to the wound site.

In a first aspect, the present disclosure provides a negative pressure wound therapy (NPWT) system for treatment of a wound site. The NPWT system includes a therapy unit including a housing, a negative pressure source disposed within the housing, and a first printed circuit board disposed within the housing. The NPWT system further includes a battery pack configured to provide operational power to the therapy unit. The battery pack is coupled to the therapy unit. The battery pack includes a second printed circuit board. The NPWT system further includes a power connector assembly configured to establish an electrical connection and a data connection between the battery pack and the therapy unit. The NPWT system further includes a wound dressing coupled to the wound site and disposed in fluid communication with the therapy unit. The NPWT system further includes an ambient pressure sensor configured to generate a signal indicative of an ambient pressure. The first printed circuit board is communicably coupled to the ambient pressure sensor for receiving the signal from the ambient pressure sensor.

In a second aspect, the present disclosure provides a negative pressure wound therapy (NPWT) system for treatment of a wound site. The NPWT system includes a therapy unit including a housing, a negative pressure source disposed within the housing, and a first printed circuit board disposed within the housing. The NPWT system further includes a battery pack configured to provide operational power to the therapy unit. The battery pack is coupled to the therapy unit. The battery pack includes a second printed circuit board. The NPWT system further includes a power connector assembly configured to establish an electrical connection and a data connection between the battery pack and the therapy unit. The NPWT system further includes a power charger configured to electrically charge the battery pack. The NPWT system further includes an ambient pressure sensor coupled to the power charger and configured to generate a signal indicative of an ambient pressure. The first printed circuit board is communicably coupled to the ambient pressure sensor for receiving the signal from the ambient pressure sensor.

In a third aspect, the present disclosure provides a method for providing negative pressure wound therapy to a wound site. The method includes providing a therapy unit including a housing, a negative pressure source disposed within the housing, and a first printed circuit board disposed within the housing. The method further includes coupling a battery pack to the therapy unit. The battery pack is configured to provide operational power to the therapy unit. The battery pack includes a second printed circuit board. The method further includes establishing, via a power connector assembly, an electrical connection and a data connection between the battery pack and the therapy unit. The method further includes fluidly communicating a wound dressing coupled to the wound site with the therapy unit. The method further includes providing an ambient pressure sensor configured to generate a signal indicative of an ambient pressure. The method further includes communicably coupling the first printed circuit board to the ambient pressure sensor for receiving the signal from the ambient pressure sensor.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 illustrates a schematic top view of a negative pressure wound therapy (NPWT) system, according to an embodiment of the present disclosure;

FIG. 2 illustrates an exploded view of a therapy unit associated with the NPWT system of FIG. 1, according to an embodiment of the present disclosure;

FIG. 3 illustrates a schematic sectional view of the therapy unit and a battery pack of the NPWT system of FIG. 1;

FIG. 4A illustrates a schematic front view of the NPWT system, according to another embodiment of the present disclosure;

FIG. 4B illustrates an exploded view of a battery pack of the NPWT system of FIG. 4A;

FIG. 5A illustrates an exploded view of a NPWT system, according to yet another embodiment of the present disclosure;

FIG. 5B illustrates a power interface associated with the NPWT system of FIG. 5A;

FIG. 6A illustrates a schematic bottom view of a portion of a therapy unit associated with a NPWT system, according to an embodiment of the present disclosure;

FIG. 6B illustrates a partial schematic sectional view of the therapy unit of FIG. 6A;

FIG. 7 illustrates a schematic top view of a NPWT system, according to another embodiment of the present disclosure;

FIG. 8 illustrates an exploded view of a power charger for a NPWT system, according to yet another embodiment of the present disclosure;

FIG. 9 illustrates an exploded view of another power charger for the NPWT system of FIG. 8, according to an embodiment of the present disclosure;

FIG. 10 illustrates a block diagram of a NPWT system and a device associated with the NPWT system, according to an embodiment of the present disclosure; and

FIG. 11 illustrates a flowchart for a method for providing negative pressure wound therapy to a wound site, according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20 % for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, “at least one of A and B” should be understood to mean “only A, only B, or both A and B”.

Unless specified or limited otherwise, the terms “attached,” “connected,” “coupled,” and variations thereof, are used broadly and encompass both direct physical connections or indirect physical connections between two or more components that are connected together by one or more additional components. For example, a first component may be coupled to a second component by being directly connected together or by being connected by a third component.

As used herein, the terms “layer,” “sheet,” and “dressing,” or variations thereof, are used to describe an article having a thickness that is small relative to its length and width.

As used herein, the term “negative pressure” broadly refers to a pressure lower than a local ambient pressure, such as an ambient pressure, in a local environment outside the sealed treatment environment provided by a dressing. In many cases, the local ambient pressure can also be the atmospheric pressure at which a wound site is located. Alternatively, the pressure can be less than the hydrostatic pressure associated with the tissue at the wound site. Unless otherwise specified, the pressure values described herein are gauge pressures. Similarly, a reference to an increase in negative pressure typically refers to a decrease in absolute pressure, while a decrease in negative pressure typically refers to an increase in absolute pressure.

As used herein, the term “wounds” can include, for example, chronic, acute, traumatic, subacute, closed surgical wounds or dehiscence wounds, partially thick burns, ulcers (such as, diabetic, compressive, or venous insufficiency ulcers), flaps, and grafts. The wound may also include an open abdomen area of a patient.

As used herein, the term “wound site” may include a tissue site, such as, bone tissue, adipose tissue, muscle tissue, nerve tissue, skin tissue, vascular tissue, connective tissue, cartilage, tendons, or ligaments. The term “wound site” may also refer to an area of a tissue that is not necessarily a wound or defect but may be desired to add or promote additional tissue growth. For example, negative pressure therapy can be used in a particular tissue area to grow additional tissue that can be harvested or transplanted to another tissue site. The wound site may also include an area wherein a surgical incision has been previously performed.

As used herein, the term “therapeutic pressure” may correlate to a target pressure prescribed for negative-pressure therapy.

A negative pressure wound therapy (NPWT) system is used to promote healing of wounds. Conventional NPWT systems typically include a therapy unit configured to provide negative pressure therapy by reducing a pressure at a wound site. Conventional therapy units may draw a vacuum at the wound site (relative to ambient pressure) by removing exudates, air, and other fluids from the wound site. The fluids removed from the wound site may be collected within an exudate canister.

Conventional therapy units include a negative pressure source, such as a pump, and a pressure sensor that provides pressure readings. However, such a pressure sensor may only be useful to administer negative pressure therapy for a short duration. For long duration negative pressure therapy applications or when users are mobile, there may be changes in ambient pressures. In such cases, an additional pressure sensor may be required. Usage of multiple pressure sensors may increase cost and complexity of the therapy device. It may be desirable to design the therapy device and incorporate the pressure sensor in the therapy device in such a way that the therapy device is compact in size, easy to use, cost-effective, and provides a sustainable solution with minimum environmental impact.

The present disclosure provides a NPWT system for treatment of a wound site. The NPWT system includes a therapy unit including a housing, a negative pressure source disposed within the housing, and a first printed circuit board disposed within the housing. The NPWT system further includes a battery pack configured to provide operational power to the therapy unit. The battery pack is coupled to the therapy unit. The battery pack includes a second printed circuit board. The NPWT system further includes a power connector assembly configured to establish an electrical connection and a data connection between the battery pack and the therapy unit. The NPWT system further includes a wound dressing coupled to the wound site and disposed in fluid communication with the therapy unit. The NPWT system further includes an ambient pressure sensor configured to generate a signal indicative of an ambient pressure. The first printed circuit board is communicably coupled to the ambient pressure sensor for receiving the signal from the ambient pressure sensor.

The therapy unit of the present disclosure may be compact in size, may be easy to use, may be cost-effective, and may have minimum environmental impact. Further, the technique of incorporating the ambient pressure sensor in the therapy unit, the battery pack, the power connector assembly, or the wound dressing may allow the therapy unit to have a hermetically sealed design, thus removing any need for internal manifolds which may otherwise increase size, cost, and material usage of the therapy unit.

Further, the present disclosure may allow usage of board-based ambient pressure sensors which are less costly, thereby reducing costs associated with the NPWT system. Thus, the present disclosure may provide a reliable means of providing ambient pressure readings to therapy units having sealed architectures to ensure therapeutic pressure is accurately maintained therein. Further, the teachings of the present disclosure may be beneficial in longer duration negative pressure therapy applications, and/or when users may experience changes in ambient pressures.

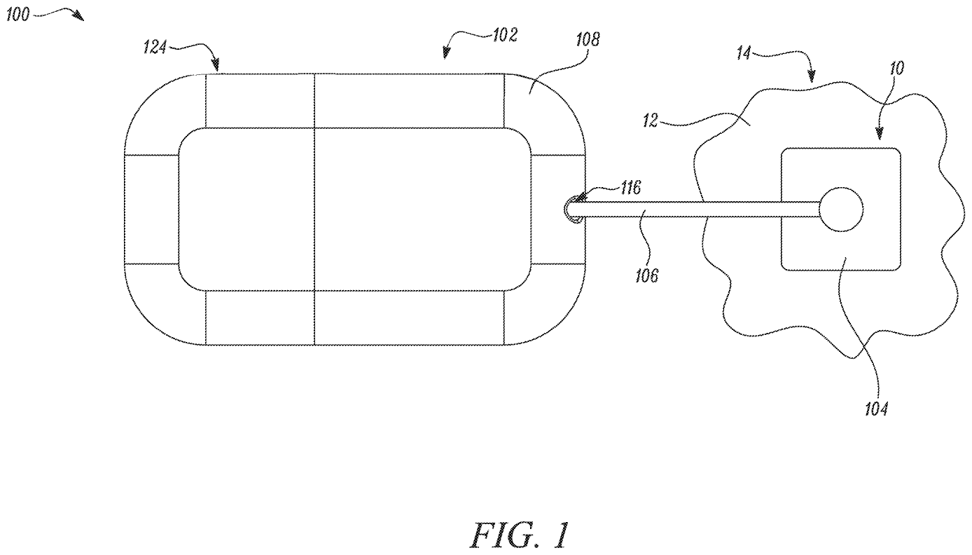

Referring now to Figures, FIG. 1 illustrates a schematic view of a NPWT system 100 for treatment of a wound site 10, according to an embodiment of the present disclosure. The NPWT system 100 may be disposed on a skin 12 of a user 14. The user 14 is a patient having a wound (not shown). The skin 12 of the user 14 includes the wound site 10. The NPWT system 100 includes a therapy unit 102. Further, the NPWT system 100 includes a wound dressing 104 coupled to the wound site 10 and disposed in fluid communication with the therapy unit 102. The wound dressing 104 encloses the wound site 10. The NPWT system 100 further includes a dressing tubing 106 that fluidly communicates the wound dressing 104 with the therapy unit 102.

The therapy unit 102 is configured to apply a negative pressure at the wound site 10. The therapy unit 102 together with the wound dressing 104 and the dressing tubing 106 may be used to apply a negative pressure wound therapy at the wound site 10. In some embodiments, the dressing tubing 106 may form an integral part of the therapy unit 102. However, the dressing tubing 106 may be replaceable, as per application requirement.

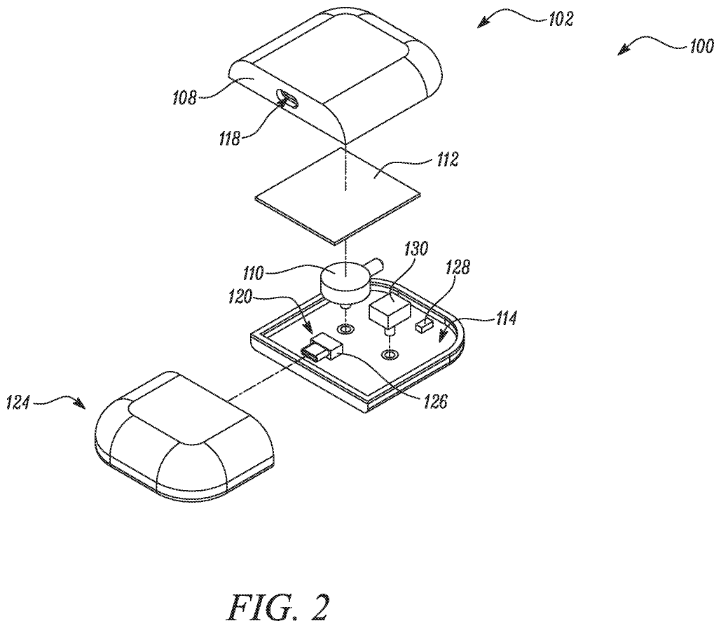

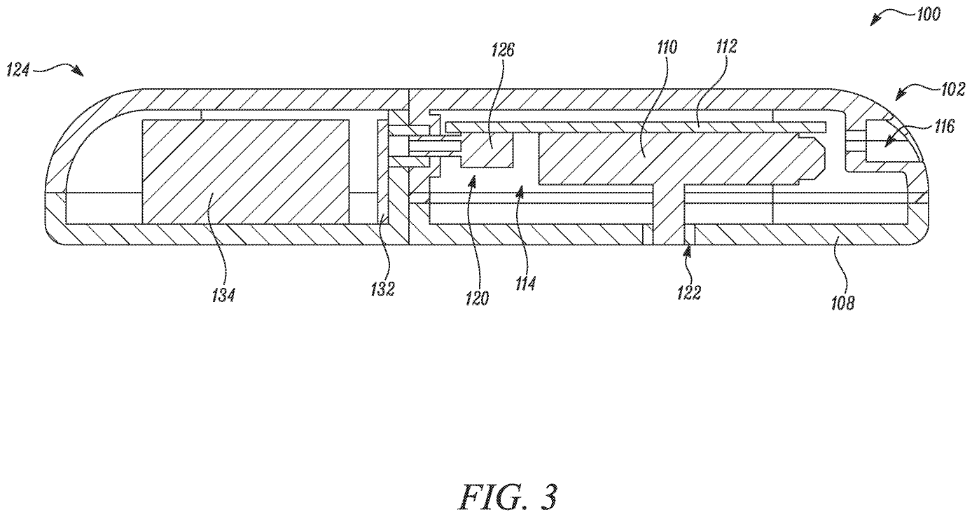

Referring to FIG. 2, the therapy unit 102 includes a housing 108, a negative pressure source 110 disposed within the housing 108, and a first printed circuit board 112 disposed within the housing 108. The housing 108 includes a hermetically sealed housing herein. In other words, the housing 108 includes a sealed architecture herein that is isolated from the ambient. In some examples, the housing 108 may be maintained at a therapeutic pressure. The housing 108 defines an interior 114 for holding components, such as, sensors, printed circuit boards or other processing units, memory/databases, etc. Further, the therapy unit 102 may include alarm indicators and/or one or more user interfaces having input and output devices. The user interface may allow users to provide an input to the therapy unit 102, for example, to initiate the negative pressure wound therapy or make some adjustments to the negative pressure wound therapy. Further, the user interface may also display information related to an ongoing negative pressure wound therapy or any other information, such as, diagnostics information related to the therapy unit 102. In some embodiments, the user interface may be embodied as a touch screen. The housing 108 defines a first opening 116 (shown in FIG. 1) that allows connection of the dressing tubing 106 (see FIG. 1) with the negative pressure source 110. The housing 108 further defines a second opening 118 that at least partially receives a portion of a power connector assembly 120. The housing 108 further defines an exhaust port 122 (shown in FIG. 3).

The negative pressure source 110 of the therapy unit 102 is configured to provide the negative pressure to the wound site 10. The negative pressure source 110 may be any device which can be operated in order to apply negative or reduced pressure to the wound site 10. The negative pressure source 110 may include an electrically powered device that can reduce pressure in a sealed volume, such as, a vacuum pump, a suction pump, or a micro-pump. The negative pressure source 110 may be housed within the therapy unit 102 and may be used in conjunction with other components, such as, sensors, printed circuit boards or processing units, alarm indicators, memory/databases, software, user interfaces, or other devices that may facilitate the negative pressure wound therapy. Further, the negative pressure applied at the wound site 10 can induce macrostrain and microstrain at the wound site 10, as well as remove exudates and other fluids from the wound site 10, which can be collected in an exudate canister (not shown) and discarded in an appropriate manner.

Further, the first printed circuit board 112 may be configured to receive input signals from the user 14 or one or more sensors associated with the therapy unit 102. Furthermore, the first printed circuit board 112 may generate one or more output signals to facilitate and control the negative pressure wound therapy. In an example, the first printed circuit board 112 may control the negative pressure source 110 to apply the negative pressure at the wound site 10. For example, the first printed circuit board 112 may control an operation of the negative pressure source 110 to adjust an amount of the negative pressure being applied. The first printed circuit board 112 may also control an activation/deactivation of the negative pressure source 110.

As shown in FIGS. 2 and 3, the NPWT system 100 further includes a battery pack 124 configured to provide operational power to the therapy unit 102. The battery pack 124 is coupled to the therapy unit 102. In the embodiments illustrated on FIGS. 2 and 3, the battery pack 124 is removably coupled to the therapy unit 102. In other embodiments, the battery pack 124 may be integral with the therapy unit 102. For example, components of the battery pack 124 and the therapy unit 102 may be disposed in a single housing. When the battery pack 124 is coupled to the therapy unit 102, the battery pack 124 and the therapy unit 102 together define a substantially cuboid-shaped device. When the therapy unit 102 is switched on, the battery pack 124 may provide the operational power to the therapy unit 102 for operation thereof. For example, the battery pack 124 may provide operational power to the negative pressure source 110. The battery pack 124 includes a second printed circuit board 132. The battery pack 124 also includes a battery module 134 that provides the operational power to the therapy unit 102. The battery module 134 may include, for example, one or more rechargeable lithium ion batteries.

The NPWT system 100 further includes the power connector assembly 120 configured to establish an electrical connection and a data connection between the battery pack 124 and the therapy unit 102. The power connector assembly 120 electrically connects the battery pack 124 with the negative pressure source 110. Further, the power connector assembly 120 communicably couples the first printed circuit board 112 and the second printed circuit board 132. In the illustrated embodiment of FIGS. 2 and 3, the power connector assembly 120 includes a power connector 126 connected to the housing 108. The power connector 126 is configured to be removably connected to the battery pack 124, such that the power connector 126 establishes the electrical connection and the data connection between the battery pack 124 and the therapy unit 102.

Referring again to FIG. 2, the NPWT system 100 includes an ambient pressure sensor 128 configured to generate a signal indicative of an ambient pressure. Further, the first printed circuit board 112 is communicably coupled to the ambient pressure sensor 128 for receiving the signal from the ambient pressure sensor 128. The ambient pressure sensor 128 may be coupled to one of the housing 108 of the therapy unit 102, the battery pack 124, the power connector assembly 120, and the wound dressing 104 (see FIG. 1).

In the illustrated embodiment of FIG. 2, the ambient pressure sensor 128 is disposed within the housing 108 of the therapy unit 102. In some embodiments, the ambient pressure sensor 128 is mounted to the first printed circuit board 112 of the therapy unit 102. Accordingly, the ambient pressure sensor 128 may be embodied as a board-based pressure sensor that may be cost-effective to incorporate. Further, the ambient pressure sensor 128 may include a barometric pressure sensor, such as, a piezo-resistive type pressure sensor. It should be noted that the present disclosure is not limited by a type of the ambient pressure sensor 128. In some embodiments, the ambient pressure sensor 128 is disposed within the housing 108 of the therapy unit 102 and is configured to measure a pressure within the housing 108 prior to an operation of the negative pressure source 110. Specifically, the ambient pressure sensor 128 is configured to measure the pressure within the housing 108 before the negative pressure source 110 is operated to maintain the housing 108 at the therapeutic pressure. This way the signals generated by the ambient pressure sensor 128 may be used to apply relative negative pressure to the wound site 10. Further, in some cases, the ambient pressure sensor 128 may be configured to measure the pressure within the housing 108 periodically due to leakage at one or more locations between the wound dressing 104 and the negative pressure source 110. For examples, leakages from the housing 108, the dressing tubing 106 (see FIG. 1), and/or the wound dressing 104 may warrant periodical measuring of the pressure within the housing 108. The technique of incorporating the ambient pressure sensor 128 in the therapy unit 102 may allow the therapy unit 102 to have a hermetically sealed design, thus removing any need for internal manifolds which may otherwise increase size, cost, and material usage of the therapy unit 102.

In some embodiments, the NPWT system 100 includes a pressure relief valve 130 coupled to the housing 108 and configured to selectively fluidly communicate the interior 114 of the housing 108 with an external ambient of the housing 108. In some examples, the pressure relief valve 130 may be configured to relieve an overpressure within the housing 108. The pressure relief valve 130 may embody any conventional relief valve that operates to relieve pressure so that gross changes in ambient pressures are not detrimental to the therapy unit 102. However, in some embodiments, the NPWT system 100 may omit the pressure relief valve 130.

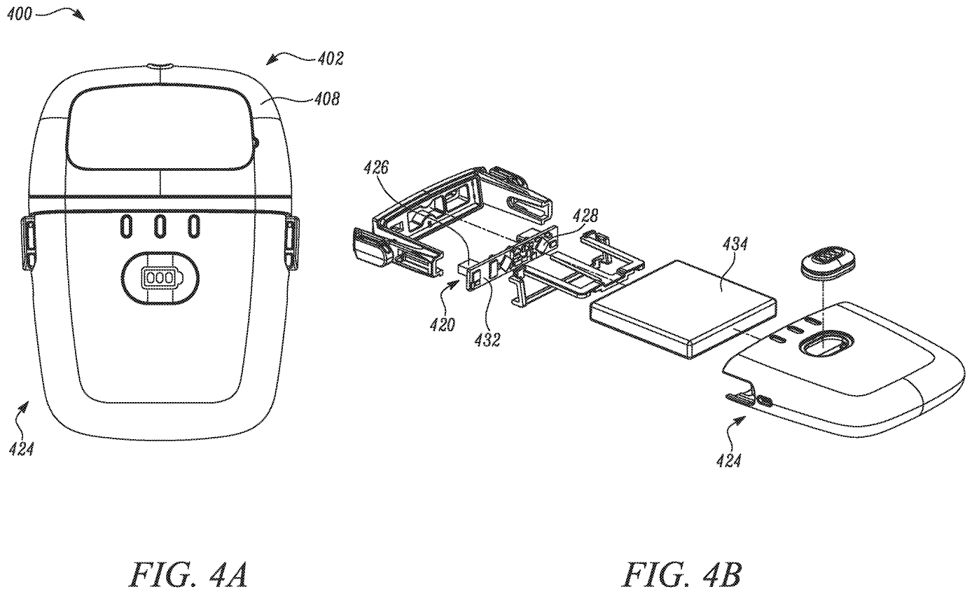

FIGS. 4A and 4B illustrates a NPWT system 400, according to another embodiment of the present disclosure. The NPWT system 400 includes a therapy unit 402 that may be functionally equivalent to the NPWT system 100 shown in FIGS. 1 to 3. Referring to FIG. 4A, the therapy unit 402 includes a housing 408, a negative pressure source (not shown) disposed within the housing 408, and a first printed circuit board (not shown) disposed within the housing 408. The negative pressure source and the first printed circuit board of the therapy unit 402 may be substantially similar and functionally equivalent to the negative pressure source 110 and the first printed circuit board 112, respectively, associated with the therapy unit 102 illustrated in FIGS. 2 and 3.

Further, the NPWT system 400 includes a battery pack 424 configured to provide operational power to the therapy unit 402. The battery pack 424 may be functionally equivalent to the battery pack 124 shown in FIGS. 1 to 3. The battery pack 424 is coupled to the therapy unit 402. In the embodiments illustrated on FIGS. 4A and 4B, the battery pack 424 is removably coupled to the therapy unit 402. In other embodiments, the battery pack 424 may be integral with the therapy unit 402. For example, components of the battery pack 424 and the therapy unit 402 may be disposed in a single housing. Referring now to FIG. 4B, the battery pack 424 includes a second printed circuit board 432. The battery pack 424 includes a battery module 434 that provides the operational power to the therapy unit 402. The battery module 434 may include, for example, one or more rechargeable lithium ion batteries.

Further, the NPWT system 400 further includes a power connector assembly 420 configured to establish an electrical connection and a data connection between the battery pack 424 and the therapy unit 402. The power connector assembly 420 includes a power connector 426. The NPWT system 400 further includes an ambient pressure sensor 428 that may be functionally equivalent to the ambient pressure sensor 128 shown in FIGS. 2 and 3. In the illustrated embodiment of FIG. 4B, the ambient pressure sensor 428 is disposed within the battery pack 424. Specifically, the ambient pressure sensor 428 is mounted to the second printed circuit board 432 of the battery pack 424. Accordingly, the ambient pressure sensor 428 may be embodied as a board-based pressure sensor that may be cost-effective to incorporate. The technique of incorporating the ambient pressure sensor 428 in the battery pack 424 may allow the therapy unit 402 to have a hermetically sealed design, thus removing any need for internal manifolds which may otherwise increase size, cost, and material usage of the therapy unit 402.

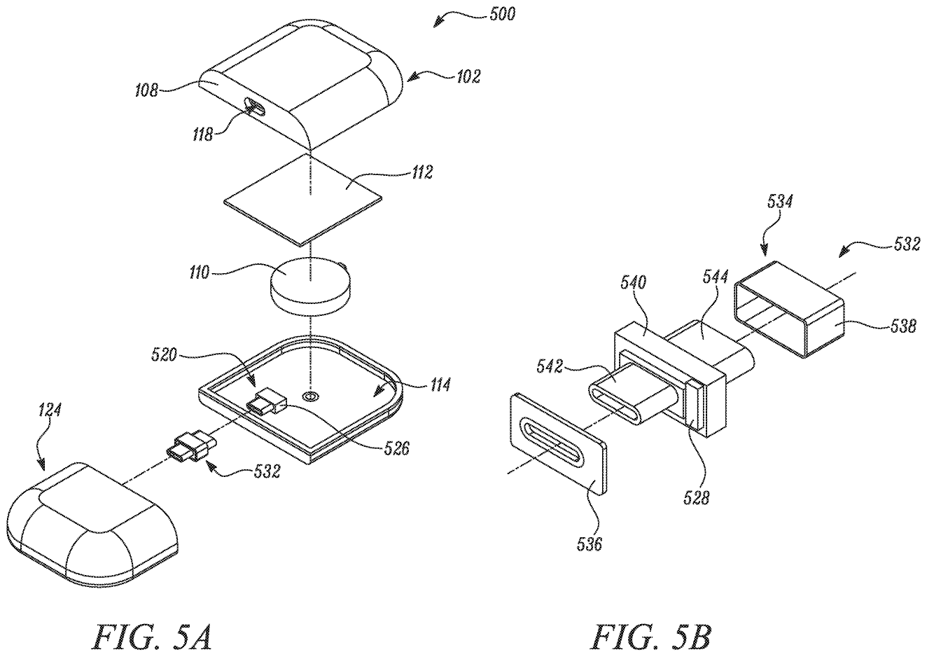

FIGS. 5A and 5B illustrate a NPWT system 500, according to yet another embodiment of the present disclosure. The NPWT system 500 is functionally equivalent to the NPWT system 100 shown in FIGS. 1 to 3. Same parts are referred to herein by same numbers. As shown in FIG. 5A, the NPWT system 500 includes the therapy unit 102, the battery pack 124, and a power connector assembly 520. The power connector assembly 520 includes a power connector 526 connected to the housing 108. The power connector assembly 520 further includes a power interface 532 configured to be removably connected to the power connector 526 and the battery pack 124, such that the power interface 532 and the power connector 526 establish an electrical connection and a data connection between the battery pack 124 and the therapy unit 102.

Referring to FIG. 5B, the power interface 532 includes a circuit board housing 534. The circuit board housing 534 includes a first housing portion 536 and a second housing portion 538. The first and second housing portions 536, 538 are in alignment with each other. Further, the power interface 532 includes a third printed circuit board 540 mounted to the circuit board housing 534. The first and second housing portions 536, 538 are disposed at either ends of the third printed circuit board 540. The power interface 532 further includes a first connecting port 542 configured to removably connect to the battery pack 124. The power interface 532 further includes a second connecting port 544 configured to removably connect to the power connector 526. The NPWT system 500 includes an ambient pressure sensor 528 that may be functionally equivalent to the ambient pressure sensor 128 shown in FIGS. 2 and 3. In the illustrated embodiment of FIG. 5B, the ambient pressure sensor 528 is mounted to the third printed circuit board 540. Accordingly, the ambient pressure sensor 528 may be embodied as a board-based pressure sensor that may be cost-effective to incorporate. The technique of incorporating the ambient pressure sensor 528 with the power connector assembly 520 may allow the therapy unit 102 to have a hermetically sealed design, thus removing any need for internal manifolds which may otherwise increase size, cost, and material usage of the therapy unit 102.

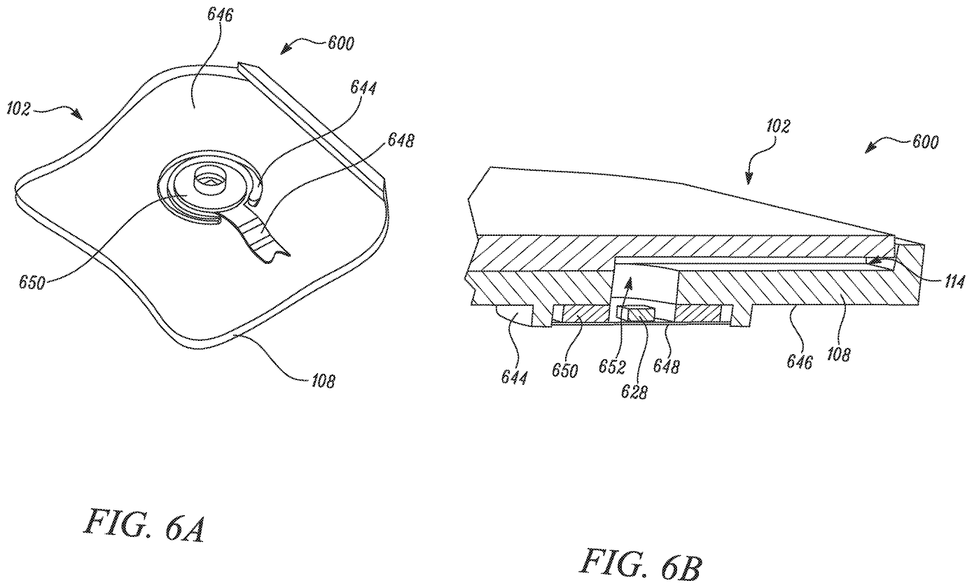

FIGS. 6A and 6B illustrate a NPWT system 600, according to an embodiment of the present disclosure. As shown in FIG. 6A, the NPWT system 600 is functionally equivalent to the NPWT system 100 shown in FIGS. 1 to 3. Same parts are referred to herein by same numbers. The NPWT system 600 includes the therapy unit 102, a battery pack (not shown), and a power connector assembly (not shown). The battery pack and the power connector assembly are functionally equivalent and similar in design to the battery pack 124 and the power connector assembly 120 as explained in relation to FIGS. 2 and 3. Further, the housing 108 of the therapy unit 102 includes an enclosure 644 extending from a lower surface 646 of the housing 108. In the illustrated embodiment of FIG. 6A, the therapy unit 102 further includes a flexible printed circuit board 648 connected to a first printed circuit board (similar to the first printed circuit board 112 of FIGS. 2 and 3) and disposed external to the housing 108.

As shown in FIG. 6B, at least a portion of the flexible printed circuit board 648 is spaced apart from the lower surface 646 of the housing 108. Further, the NPWT system 600 includes an ambient pressure sensor 628. The ambient pressure sensor 628 may be functionally equivalent to the ambient pressure sensor 128 shown in FIGS. 2 and 3. In the illustrated embodiment of FIG. 6B, the ambient pressure sensor 628 is mounted to the flexible printed circuit board 648. In other words, the ambient pressure sensor 628 is mounted outside the housing 108, in the ambient, and on the flexible printed circuit board 648. Further, the ambient pressure sensor 628 may be embodied as a board-based pressure sensor that may be cost-effective to incorporate.

The NPWT system 600 further includes a sealing enclosure 650 disposed between the flexible printed circuit board 648 and the housing 108. The sealing enclosure 650 is configured to surround the ambient pressure sensor 628. The sealing enclosure 650 is annular in shape and is configured to accommodate the ambient pressure sensor 628 therein. Further, a space within the sealed enclosure 650 communicates with an opening 652 provided in the housing 108. The sealing enclosure 650 may sealingly connected to and may extend between the lower surface 646 of the housing 108 and the flexible printed circuit board 648. The technique of incorporating the ambient pressure sensor 628 on the flexible printed circuit board 648, and external to the housing 108, may allow the therapy unit 102 to have a hermetically sealed design, thus removing any need for internal manifolds which may otherwise increase size, cost, and material usage of the therapy unit 102.

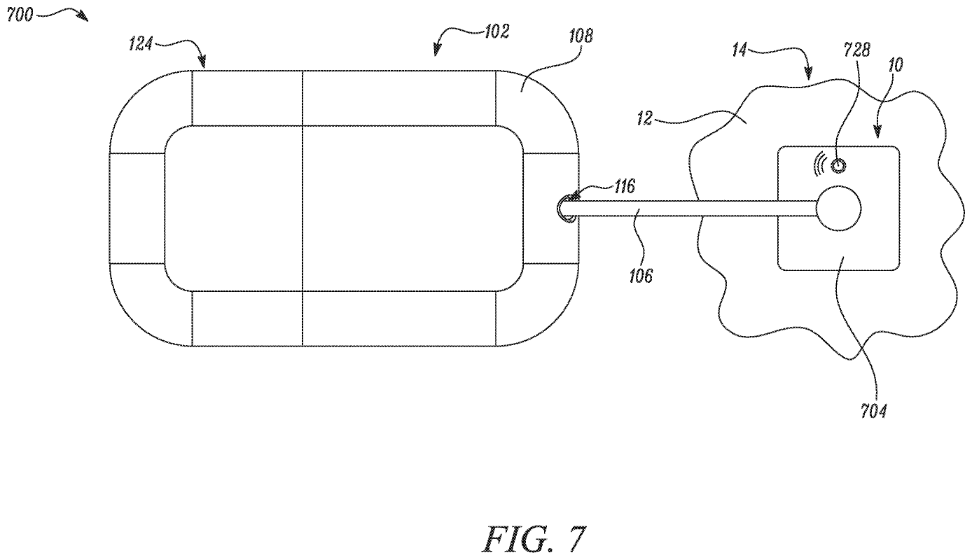

FIG. 7 illustrates a NPWT system 700, according to another embodiment of the present disclosure. The NPWT system 700 is functionally equivalent to the NPWT system 100 shown in FIGS. 1 to 3. Same parts are referred to herein by same numbers. The NPWT system 700 includes a wound dressing 704, the dressing tubing 106, the therapy unit 102, and the battery pack 124. The wound dressing 704 may be functionally equivalent to the wound dressing 104 shown in FIG. 1. The NPWT system 700 further includes an ambient pressure sensor 728 that may be functionally equivalent to the ambient pressure sensor 128 shown in FIGS. 2 and 3. In the illustrated embodiment of FIG. 7, the ambient pressure sensor 728 is disposed within the wound dressing 704. In some embodiments, the wound dressing 704 may embody a 3M™ instrumented T.R.A.C.™ Pad. The ambient pressure sensor 728 may be wirelessly connected to a first printed circuit board (similar to the first printed circuit board 112) of the therapy unit 102, such as, via Bluetooth or Wi-Fi, without any limitations. Further, the technique of incorporating the ambient pressure sensor 728 in the wound dressing 704 may allow the therapy unit 102 to have a hermetically sealed design, thus removing any need for internal manifolds which may otherwise increase size, cost, and material usage of the therapy unit 102.

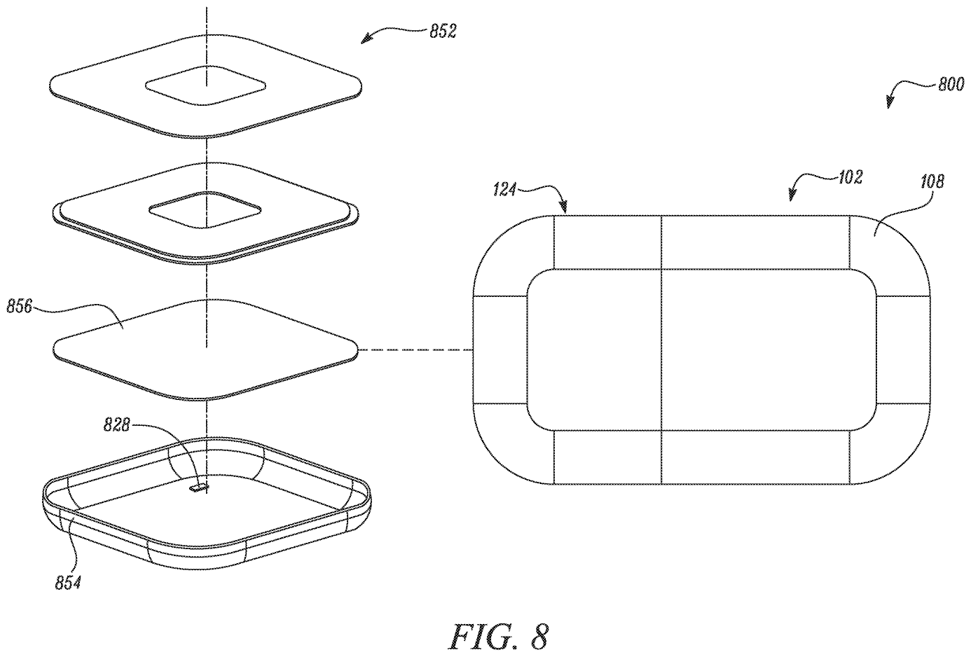

FIG. 8 illustrates a NPWT system 800, according to yet another embodiment of the present disclosure. The NPWT system 800 is functionally equivalent to the NPWT system 100 shown in FIGS. 1 to 3. Same parts are referred to herein by same numbers. The NPWT system 800 includes the therapy unit 102, the battery pack 124, and a power charger 852 configured to electrically charge the battery pack 124. Further, the NPWT system 800 also includes a power connector assembly (not shown) that is functionally equivalent and similar in design to the power connector assembly 120 as explained in relation to FIGS. 2 and 3.

The NPWT system 800 further includes an ambient pressure sensor 828 coupled to the power charger 852. The ambient pressure sensor 828 is configured to generate a signal indicative of an ambient pressure. Further, the first printed circuit board 112 is communicably coupled to the ambient pressure sensor 828 for receiving the signal from the ambient pressure sensor 828.

In the illustrated embodiment of FIG. 8, the power charger 852 includes a wireless charger 852 including a casing 854. The power charger 852 will be hereinafter interchangeably referred to as the wireless charger 852. Further, the power charger 852 includes a printed circuit board 856 disposed within the casing 854. The ambient pressure sensor 828 is disposed within the casing 854 of the wireless charger 852. In some embodiments, the ambient pressure sensor 828 may be mounted to the printed circuit board 856 of the power charger 852. Further, the technique of incorporating the ambient pressure sensor 828 in the wireless charger 852 may allow the therapy unit 102 to have a hermetically sealed design, thus removing any need for internal manifolds which may otherwise increase size, cost, and material usage of the therapy unit 102.

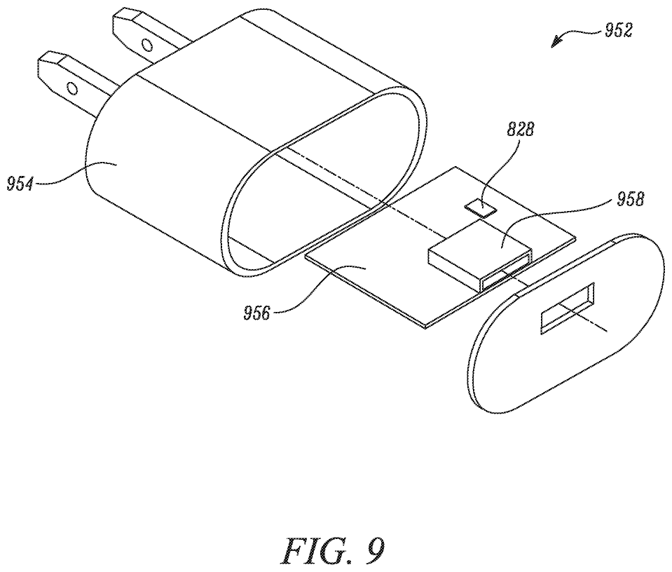

FIG. 9 illustrates another power charger 952 that may be used to charge the battery pack 124 of the NPWT system 800 shown in FIG. 8. The power charger 952 may connect with an alternating current (AC) mains electricity power supply. In the illustrated embodiment of FIG. 9, the power charger 952 includes a universal serial bus (USB) connector 958 configured to be removably coupled to the battery pack 124. Further, a connecting cable (not shown) may removably couple the USB connector 958 with the battery pack 124 to establish an electrical connection between the USB connector 958 and the battery pack 124. The power charger 952 includes a printed circuit board 956. The USB connector 958 may be disposed within a casing 954 of the power charger 952. In the illustrated embodiment of FIG. 9, the ambient pressure sensor 828 is mounted to the printed circuit board 956. Accordingly, the ambient pressure sensor 828 may be embodied as a board-based pressure sensor that may be cost-effective to incorporate. Further, the technique of incorporating the ambient pressure sensor 828 in the power charger 952 may allow the therapy unit 102 to have a hermetically sealed design, thus removing any need for internal manifolds which may otherwise increase size, cost, and material usage of the therapy unit 102.

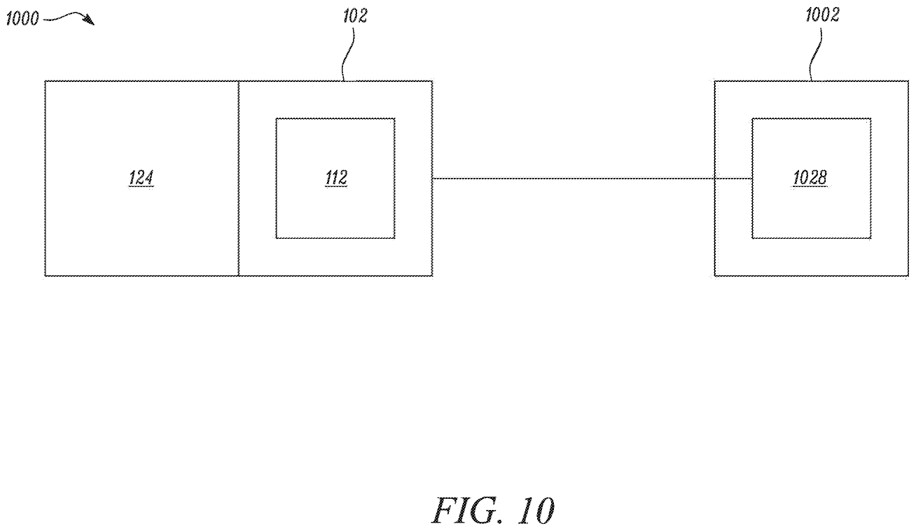

FIG. 10 illustrates a NPWT system 1000, according to an embodiment of the present disclosure. The NPWT system 1000 is functionally equivalent to the NPWT system 100 shown in FIGS. 1 to 3. Same parts are referred to herein by same numbers. The NPWT system 1000 includes the therapy unit 102, the first printed circuit board 112, and the battery pack 124. The NPWT system 1000 further includes an ambient pressure sensor 1028. The ambient pressure sensor 1028 is configured to generate a signal indicative of an ambient pressure.

In the illustrated embodiment of FIG. 10, the NPWT system 1000 includes a device 1002 wirelessly communicating with the first printed circuit board 112. The ambient pressure sensor 1028 is disposed within the device 1002. The ambient pressure sensor 1028 may embody a barometric pressure sensor capable of measuring ambient pressure readings. The device 1002 may include a mobile/cell phone, a tablet, a laptop, and the like having the ambient pressure sensor 1028 associated therewith. It should be noted that the device 1002 may include any electronic device having an inbuilt pressure sensor capable of measuring ambient pressure readings. In some examples, the device 1002 may embody a Bluetooth-enabled device having the ambient pressure sensor 1028. In such examples, the device 1002 may form a part of a kit including the therapy unit 102 and the battery pack 124. It should be noted that the present disclosure is not limited by a type of the device 1002.

Further, the first printed circuit board 112 is communicably coupled to the ambient pressure sensor 1028 for receiving the signal from the ambient pressure sensor 1028. The ambient pressure sensor 1028 may be wirelessly connected to the first printed circuit board 112 of the therapy unit 102, such as, via Bluetooth or Wi-Fi, without any limitations.

Referring to FIGS. 1 to 10, the NPWT system 100, 400, 500, 600, 700, 800, 1000 of the present disclosure may be compact in size, may be easy to use, may be cost-effective, and may have minimum environmental impact. Further, the present disclosure may also allow usage of board-based ambient pressure sensors which are less costly, thereby reducing a cost associated with the NPWT system 100, 400, 500, 600, 700, 800, 1000. Thus, the present disclosure may provide a reliable means of providing ambient pressure readings to the therapy units 102, 402 having sealed architectures to ensure therapeutic pressures are accurately maintained therein. Further, the teachings of the present disclosure may be beneficial in longer duration negative pressure therapy applications, and/or when the user 14 may experience changes in ambient pressures.

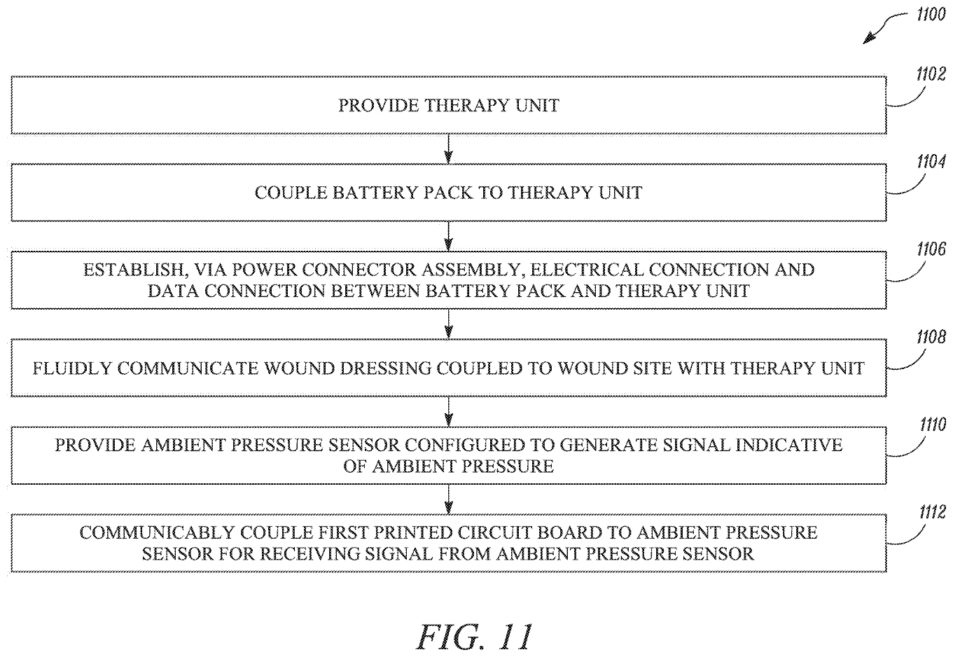

FIG. 11 illustrates a flowchart depicting a method 1100 for providing the negative pressure wound therapy to the wound site 10. Referring to FIGS. 1 to 7, and FIGS. 10-11, at step 1102, the method 1100 includes providing the therapy unit 102, 402 including the housing 108, 408, the negative pressure source 110, 410 disposed within the housing 108, 408, and the first printed circuit board 112 disposed within the housing 108, 408. At step 1104, the method 1100 includes coupling the battery pack 124, 424 to the therapy unit 102, 402. The battery pack 124, 424 is configured to provide the operational power to the therapy unit 102, 402. Further, the battery pack 124, 424 includes the second printed circuit board 132, 432.

At step 1106, the method 1100 includes establishing the electrical connection and the data connection between the battery pack 124, 424 and the therapy unit 102, 402 via the power connector assembly 120, 420. At step 1108, the method 1100 includes fluidly communicating the wound dressing 104 coupled to the wound site 10 with the therapy unit 102, 402. Further, at step 1110, the method 1100 includes providing the ambient pressure sensor 128, 428, 528, 628, 728, 1028 configured to generate the signal indicative of the ambient pressure. In some embodiments, the method 1100 further includes coupling the ambient pressure sensor 128, 428, 528, 628, 728, 1028 to one of the housing 108 of the therapy unit 102, the battery pack 424, the power connector assembly 520, and the wound dressing 704.

Referring to FIGS. 2, 3, and 11, in some embodiments, the housing 108 includes the hermetically sealed housing. Further, at the step 1110, the coupling of the ambient pressure sensor 128 to the housing 108 of the therapy unit 102 further includes disposing the ambient pressure sensor 128 within the housing 108 of the therapy unit 102. Further, the method 1100 includes measuring, via the ambient pressure sensor 128, the pressure within the housing 108 prior to the operation of the negative pressure source 110. In some embodiments, the method 1100 further includes coupling the pressure relief valve 130 to the housing 108. The pressure relief valve 130 is configured to selectively fluidly communicate the interior 114 of the housing 108 with the external ambient of the housing 108. Further, the method 1100 includes relieving, via the pressure relief valve 130, the overpressure within the housing 108.

Referring to FIGS. 5A, 5B, and 11, in some embodiments, the power connector assembly 520 includes the power connector 526 connected to the housing 108 and the power interface 532 configured to be removably connected to the power connector 526 and the battery pack 124, such that the power interface 532 and the power connector 526 establish the electrical connection and the data connection between the battery pack 124 and the therapy unit 102. Further, at the step 1110, the coupling of the ambient pressure sensor 528 to the power connector assembly 520 further includes mounting the ambient pressure sensor 528 to the third printed circuit board 540 of the power interface 532.

Referring to FIGS. 6A, 6B, and 11, in some embodiments, the therapy unit 102 further includes the flexible printed circuit board 648 connected to the first printed circuit board 112 and disposed external to the housing 108. Further, at the step 1110, the coupling of the ambient pressure sensor 628 to the housing 108 of the therapy unit 102 further includes mounting the ambient pressure sensor 628 to the flexible printed circuit board 648.

Referring to FIGS. 10 and 11, the NPWT system 1000 further includes a device 1002 wirelessly communicating with the first printed circuit board 112. Further, the method 1100 further includes disposing the ambient pressure sensor 1028 within the device 1002.

Referring to FIGS. 1 to 7, and FIGS. 10-11, at step 1112, the method 1100 includes communicably coupling the first printed circuit board 112 to the ambient pressure sensor 128, 428, 528, 628, 728, 1028 for receiving the signal from the ambient pressure sensor 128, 428, 528, 628, 728, 1028.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A negative pressure wound therapy (NPWT) system for treatment of a wound site, the NPWT system comprising:
a therapy unit comprising a housing, a negative pressure source disposed within the housing, and a first printed circuit board disposed within the housing;
a battery pack configured to provide operational power to the therapy unit, wherein the battery pack is coupled to the therapy unit, and wherein the battery pack comprises a second printed circuit board;
a power connector assembly configured to establish an electrical connection and a data connection between the battery pack and the therapy unit;
a wound dressing coupled to the wound site and disposed in fluid communication with the therapy unit; and
an ambient pressure sensor configured to generate a signal indicative of an ambient pressure, and wherein the first printed circuit board is communicably coupled to the ambient pressure sensor for receiving the signal from the ambient pressure sensor.

2. The NPWT system of claim 1, wherein the ambient pressure sensor is coupled to one of the housing of the therapy unit, the battery pack, the power connector assembly, and the wound dressing.

3. The NPWT system of claim 2, wherein the housing comprises a hermetically sealed housing, and wherein the ambient pressure sensor is disposed within the housing of the therapy unit and is configured to measure a pressure within the housing prior to an operation of the negative pressure source, and the ambient pressure sensor is mounted to the first printed circuit board of the therapy unit.

4. (canceled)

5. The NPWT system of claim 2, further comprising a pressure relief valve coupled to the housing and configured to selectively fluidly communicate an interior of the housing with an external ambient of the housing.

6. The NPWT system of claim 2, wherein the ambient pressure sensor is disposed within the battery pack, and wherein the ambient pressure sensor is mounted to the second printed circuit board of the battery pack.

7. The NPWT system of claim 2, wherein the power connector assembly comprises a power connector connected to the housing and a power interface configured to be removably connected to the power connector and the battery pack, such that the power interface and the power connector establish the electrical connection and the data connection between the battery pack and the therapy unit, the power interface comprising:
a circuit board housing;
a third printed circuit board mounted to the circuit board housing;
a first connecting port configured to removably connect to the battery pack; and
a second connecting port configured to removably connect to the power connector.

8. The NPWT system of claim 7, wherein the ambient pressure sensor is mounted to the third printed circuit board.

9. The NPWT system of claim 2, wherein the therapy unit further comprises a flexible printed circuit board connected to the first printed circuit board and disposed external to the housing, and the ambient pressure sensor is mounted to the flexible printed circuit board.

10. (canceled)

11. The NPWT system of claim 9, further comprising a sealing enclosure disposed between the flexible printed circuit board and the housing, wherein the sealing enclosure is configured to surround the ambient pressure sensor.

12. The NPWT system of claim 2, wherein the ambient pressure sensor is disposed within the wound dressing.

13. (canceled)

14. A negative pressure wound therapy (NPWT) system for treatment of a wound site, the NPWT system comprising:
a therapy unit comprising a housing, a negative pressure source disposed within the housing, and a first printed circuit board disposed within the housing;
a battery pack configured to provide operational power to the therapy unit, wherein the battery pack is coupled to the therapy unit, and wherein the battery pack comprises a second printed circuit board;
a power connector assembly configured to establish an electrical connection and a data connection between the battery pack and the therapy unit;
a power charger configured to electrically charge the battery pack; and
an ambient pressure sensor coupled to the power charger and configured to generate a signal indicative of an ambient pressure, wherein the first printed circuit board is communicably coupled to the ambient pressure sensor for receiving the signal from the ambient pressure sensor.

15. The NPWT system of claim 14, wherein the power charger comprises a wireless charger comprising a casing, and wherein the ambient pressure sensor is disposed within the casing of the wireless charger.

16. The NPWT system of claim 14, wherein the power charger comprises a universal serial bus (USB) connector configured to be removably coupled to the battery pack.

17. The NPWT system of claim 16, wherein the power charger comprises a printed circuit board, and wherein the ambient pressure sensor is mounted to the printed circuit board.

18. A method for providing negative pressure wound therapy to a wound site, the method comprising:
providing a therapy unit comprising a housing, a negative pressure source disposed within the housing, and a first printed circuit board disposed within the housing;
coupling a battery pack to the therapy unit, wherein the battery pack is configured to provide operational power to the therapy unit, and wherein the battery pack comprises a second printed circuit board;
establishing, via a power connector assembly, an electrical connection and a data connection between the battery pack and the therapy unit;
fluidly communicating a wound dressing coupled to the wound site with the therapy unit;
providing an ambient pressure sensor configured to generate a signal indicative of an ambient pressure; and
communicably coupling the first printed circuit board to the ambient pressure sensor for receiving the signal from the ambient pressure sensor.

19. The method of claim 18, further comprising coupling the ambient pressure sensor to one of the housing of the therapy unit, the battery pack, the power connector assembly, and the wound dressing.

20. The method of claim 19, wherein the housing comprises a hermetically sealed housing, wherein coupling the ambient pressure sensor to the housing of the therapy unit further comprises:
disposing the ambient pressure sensor within the housing of the therapy unit; and
measuring, via the ambient pressure sensor, a pressure within the housing prior to an operation of the negative pressure source.

21. The method of claim 19, further comprising:
coupling a pressure relief valve to the housing, wherein the pressure relief valve is configured to selectively fluidly communicate an interior of the housing with an external ambient of the housing; and
relieving, via the pressure relief valve, an overpressure within the housing.

22. The method of claim 19, wherein the power connector assembly comprises a power connector connected to the housing and a power interface configured to be removably connected to the power connector and the battery pack, such that the power interface and the power connector establish the electrical connection and the data connection between the battery pack and the therapy unit, and wherein coupling the ambient pressure sensor to the power connector assembly further comprises mounting the ambient pressure sensor to a third printed circuit board of the power interface.

23. The method of claim 19, wherein the therapy unit further comprises a flexible printed circuit board connected to the first printed circuit board and disposed external to the housing, and wherein coupling the ambient pressure sensor to the housing of the therapy unit further comprises mounting the ambient pressure sensor to the flexible printed circuit board.

24. (canceled)
